(19) United States
(12) Patent Application Publication
(10) Pub. No.: US 2010/0178360 A1
(43) Pub. Date: Jul. 15, 2010

(54) NUTRITIVE COMPOSITION

(75) Inventors: Jacques Deviere, Charleroi (BE); Nicolas Cauche, Bruxelles (BE); Alain Delchambre, Bruxelles (BE); Marianna Arvanitaki, La Hulpe (BE)

Correspondence Address: MERCHANT & GOULD PC, P.O. BOX 2903, MINNEAPOLIS, MN, 55402-0903, US

(73) Assignee: Universite Libre de Bruxelles, Bruxelles (BE)

(21) Appl. No.: 12/633,405

(22) Filed: Dec. 8, 2009

(30) Foreign Application Priority Data
Jan. 13, 2009 (EP) 09150475.3

(51) Int. Cl. A61K 33/34; A61K 31/715; A61K 33/00

(52) U.S. Cl. 424/638; 514/54; 424/722

(57) ABSTRACT

An energetic or nutritive composition includes at least one compound having laxative properties, mineral salts (or electrolytes) such as sodium, potassium, chlorine, calcium, magnesium, phosphor. The composition may also include carbohydrates that may be assimilated by the human digestive system such as glucose, galactose, fructose, maltose, saccharose, polysaccharides.

### FIELD OF THE INVENTION

The present invention relates to an energetic or nutritive composition intended for preparing the digestive tract prior to a physical check-up such as an endoscopic examination (flexible endoscopy or endoscopic capsule), radiological or a therapeutic intervention.

### STATE OF THE ART

Prior to an examination of the digestive tract, it is absolutely necessary that the latter be cleaned of stools which obstruct it in order to allow adequate viewing of the mucosa.

Several classifications of the quality of the preparation have been established. (Eliakim et al. Evaluation of the PillCam Colon capsule in the detection of colonic pathology: results of the first multicenter, prospective, comparative study. Endoscopy 2006; 38(10):963-970,
Vanner et al. A randomized prospective trial comparing oral sodium phosphate with standard polyethylene glycol-based lavage solution (Golytely) in the preparation of patients for colonoscopy. American Journal of Gastroenterology 1990; 85:422-427,
Barclay et al. Carbohydrate-electrolyte re-hydration protects against intravascular volume contraction during colonic cleansing with orally administered sodium phosphate. Gastrointestinal Endoscopy 2002; 56:633-8). According to Eliakim et al., it is possible to classify the preparation in different grades, from grade 1 (excellent preparation) to grade 4 (insufficient preparation compromising the quality of the examination). Vanner et al. propose another classification described hereafter: excellent preparation=small volume of clear liquid; good preparation=important volume of clear liquid; acceptable preparation=limited half-solid stools which can be sucked up or cleaned; insufficient preparation=half-solid stools.

Poor preparation may therefore be detrimental to the quality of an examination, in particular in the case of an examination by flexible endoscopy, but also to the quality of a radiological examination where stools may mimic the presence of polyps, or in case of an examination by endoscopic capsule where the quality of the preparation has to be excellent since the capsule navigates in the liquid contained in the digestive tract which has to be perfectly clear (transparent) in order to allow viewing of the mucosa.

In case of such an examination by endoscopic capsule, insufflation or suction of debris and/or of turbid liquid cannot be authorized, whereas the physician can actively intervene when a coloscope is used (e.g. suction to clean, go back). The latter represents a reason why in case of examination by endoscopic capsule, the quality of the preparation has to be excellent. Upon use of endoscopic capsules the digestive tract thus has to be prepared better than when using a coloscope to obtain the same quality of images. This is confirmed in the abstract of ‘Capsule Endoscopy versus Colonoscopy for the Detection of Polyps and Cancer’, N Engl J Med 2009; 361:264-70.

All the procedures using colic compositions for flexible endoscopy or radiological examination (coloscan) foresee a diet without residues and administration of a laxative composition on the day before and/or the day of the examination.

In order to obtain an examination of maximum quality, a clear liquid diet (a clear liquid diet consists of the intake of transparent liquid foodstuffs such as water, defatted broth, fruit juices without pulps (apples, white grape), clear lemonades, clear gelatin, coffee (without milk), tea (without milk), herbal teas) may be recommended during the whole day preceding the examination.

The quality of the examination might further be improved if this clear liquid diet is also associated with the intake of laxative(s) during the 36 hours preceding the conduction of the examination.

This type of prolonged administration of laxatives causes an important discomfort for the patient which is increased by the intake of a large amount of liquid together with a lack of supply of nutritive elements. This may give rise to lipothymias, malaise, nauseas related to fasting, altering the comfort of the patient and therefore his/her availability upon conducting the examination but therefore also lowering patient compliance with the diet prescribed by their physician in preparation of the check-up. All of the above can give rise to a suboptimal preparation and thus less reliable results, images for review and diagnosis. Further, laxatives have a salty taste which makes their ingurgitation unpleasant.

Polyethylene glycol (PEG) represents the basis of most colic compositions, its macromolecules not being absorbed by the digestive tract, allow an increase in the volume of intestinal liquids. Accordingly, better hydrated stools transit faster in the colon and are more easily evacuated. Other laxatives such as sodium phosphates may also be used.

Presently there are several laxative or colic compositions on the market: Colofort® (Ipsen), Colopeg® (Bayer), Endofalk® (Codali), Fleet® Phosphosoda (Kela), Klean-Prep® (Norgine), etc. Some of them attempt to solve the problem of unpleasant taste by adding aromas.

In document US-A-20060073214, with the composition and/or the preparation method, the ingestion of large amounts of liquid tolerated with difficulty by the patient may be limited.

The ideal preparation method would reliably empty the colon of all fecal material, have little effect on the gross or the microscopic appearance of the mucosa, require a relatively short period for ingestion and evaluation, cause little patient discomfort, and produce no significant fluid-electrolyte shifts while maximizing the detection of colonic disease. It becomes important to consider, in particular in the field of capsule endoscopy, to be able to give nutrients while preparations for long durations can be contemplated which remove any opaque liquid residue at colon level.

### SUMMARY OF THE INVENTION

The present invention relates to an energetic or nutritive composition comprising:
at least one compound having laxative properties;
mineral salts (or electrolytes) comprising sodium, potassium, chlorine, calcium, magnesium, phosphor; and/or
carbohydrates which may be assimilated by the human digestive system such as glucose, galactose, fructose, maltose, saccharose, polysaccharides.

Preferably, vitamins such as ascorbic acid can also be added to the energetic or nutritive composition.

Preferably, the amount of such vitamins and in particular ascorbic acid is lower than 10 g.

Preferably, the energetic or nutritive composition has an energy value larger than or equal to 250 kcal.

Preferably, the energetic or nutritive composition has an energy value larger than or equal to 500 kcal.

Preferably, the energetic or nutritive composition has an energy value larger than or equal to 1000 kcal.

Preferably, the compound having laxative properties belongs to the class of osmotic laxatives.

Preferably, the compound having laxative properties is polyethylene glycol (PEG) the average molecular weight of which is larger than or equal to 3000 kDa.

Preferably, the compound having laxative properties is polyethylene glycol (PEG) the average molecular weight of which is comprised between 3000 kDa and 20000 kDa.

Preferably, the compound having laxative properties is polyethylene glycol (PEG) the average molecular weight of which is 3350 kDa.

Preferably, the energetic or nutritive composition comprises trace elements.

Preferably, the energetic or nutritive composition comprises proteins.

The present invention also relates to a kit comprising:
either at least one sachet or flask containing the composition according to the present invention,
or at least one sachet or flask containing a laxative, at least one sachet or flask containing one or more mineral salts and at least one sachet or flask containing carbohydrates,
   or at least one sachet or flask containing a laxative and at least one sachet or flask containing one or more mineral salts and carbohydrates.

The invention relates to an energetic or nutritive composition comprising at least one compound having laxative properties and nutrients.

The nutritive composition according to the present invention is transparent (clear).

Preferentially, the compounds having laxative properties are selected from the following classes of compounds: ballast laxatives, lubricants, osmotic laxatives, contact laxatives, anthraquinone derivatives, diphenylmethane derivatives and/or a mixture of these compounds.

Preferentially, the compounds having laxative properties belong to the class of osmotic laxatives.

Preferentially, the compound having laxative properties is polyethylene glycol having a molecular weight greater than or equal to 3000 kDa.

Preferentially, the compound having laxative properties is polyethylene glycol 3350.

The nutrients comprise carbohydrates which can be assimilated by the human digestive system such as glucose, maltose, galactose, fructose, saccharose, polysaccharides, etc.

The nutrients comprise mineral salts (or electrolytes) such as sodium, potassium, chlorine, calcium, magnesium salts, etc.

Preferentially, the nutrients further comprise trace elements.

Preferentially, the nutrients further comprise proteins which respect the perfectly transparent (clear) character of the administered liquid, i.e. the transparency to visible light.

Preferentially, said energetic or nutritive composition further comprises at least one food additive.

Preferentially, one of the food additives is a sweetener.

Preferentially, one of the additives is a food additive having a taste-correcting effect.

Preferentially, one of the food additives is a flavoring.

Said flavorings may be present in variable quantity and may be adapted to everyone's taste.

Preferentially, said energetic or nutritive composition is packaged in a unique sachet.

Alternatively, said energetic or nutritive composition is packaged in at least two sachets.

Alternatively, said energetic or nutritive composition is conditioned in at least one sachet and at least one container for liquids (flask).

Alternatively, said energetic or nutritive composition is conditioned in at least one container for liquids (flask).

Preferentially, this composition is a powder and/or a liquid solution.

Preferentially, this composition is used for preparing a liquid solution.

Preferentially, this liquid solution is obtained by mixing the composition according to said invention and a solvent.

Preferentially, said solvent is water.

Preferentially, administration of said solution is performed orally.

Alternatively, this composition may be found as gelatin capsules or tablets with enteric coating.

Alternatively, this composition may combine the aforementioned administration forms.

The composition according to the present invention has an energy value greater than or equal to 250 kcal.

Preferentially, said composition has an energy value greater than or equal to 500 kcal.

Preferentially, said composition has an energy value greater than or equal to 1000 kcal.

The present invention also relates to a kit assembly comprising:
either at least one sachet or one flask containing the energetic nutritive composition,
or at least one sachet or flask containing a laxative, at least one sachet or flask containing one or more mineral salts and at least one sachet or flask containing carbohydrates, forming a composition according to the present invention.
or at least one sachet or flask containing a laxative and at least one sachet or flask containing one or more mineral salts and carbohydrates, forming a composition according to the present invention.

### DETAILED DESCRIPTION OF THE INVENTION

### Composition 1:

This composition, made for a person of less than 80 kg, exists as a powder to be diluted in water up to 1.5 liters.

This composition is packaged in a single sachet comprising:

| Energy value: | 667 | kcal |
|---|---|---|
| NaCl | 2.19 | g |
| Na₂SO₄ anh. | 8.52 | g |
| KCl | 2 g ≦ KCl ≦ 4.5 g | |
| NaHCO3 | 2.52 | g |
| PEG 3350 | 88.5 | g |
| Carbohydrates | 167.4 | g |
| Glucose | 5.4 | g |
| Maltose | 20.79 | g |
| Polysaccharides | 139.86 | g |
| Electrolytes | | |
| Ca²⁺ | 0.06 g ≦ Ca²⁺ ≦ 0.16 g | |
| Mg²⁺ | 0.032 g ≦ Mg²⁺ ≦ 0.13 g | |
| PO₄³⁻ | 0.2 g ≦ PO₄³⁻ ≦ 0.4 g | |
| Food additive | | |
| Citric acid | 1.35 | g |
| Proteins | 20 | g |
| Trace elements | | |
| Iron | 3.33 mg ≦ Fe ≦ 5 mg | |
| Zinc | 3 mg ≦ Zn ≦ 4.67 mg | |
| Copper | 0.5 mg ≦ Cu ≦ 0.67 mg | |
| Manganese | 0.67 mg ≦ Mn ≦ 1.67 mg | |
| Fluorine | 0.5 mg ≦ F ≦ 1.33 mg | |
| Cobalt | ≦0.05 | μg |
| Selenium | ≦23.33 | μg |

| | |
|---|---|
| Chromium | 16.67 μg ≦ Cr ≦ 66.67 μg |
| Molybdenum | 10 μg ≦ Mo ≦ 33.33 μg |

### Composition 2:

This composition made for a person of less than 80 kg, exists as a powder to be diluted in water up to 1.5 liters.

This composition is conditioned in two sachets comprising:

Sachet 1

| | |
|---|---|
| NaCl | 2.19 g |
| Na₂SO₄ anh. | 8.52 g |
| KCl | 2 g ≦ KCl ≦ 4.5 g |
| NaHCO3 | 2.52 g |
| PEG 3350 | 88.5 g |

Sachet 2

| Energy value | 667 | kcal |
|---|---|---|
| carbohydrates | 167.4 | g |
| Glucose | 5.4 | g |
| Maltose | 20.79 | g |
| Polysaccharides | 139.86 | g |
| Electrolytes | | |
| Ca²⁺ | 0.06 g ≦ Ca²⁺ ≦ 0.16 g | |
| Mg²⁺ | 0.032 g ≦ Mg²⁺ ≦ 0.13 g | |
| PO₄³⁻ | 0.2 g ≦ PO₄³⁻ ≦ 0.4 g | |
| Food additive | | |
| Citric acid | 1.35 | g |
| Proteins | 20 | g |
| Trace elements | | |
| Iron | 3.33 mg ≦ Fe ≦ 5 mg | |
| Zinc | 3 mg ≦ Zn ≦ 4.67 mg | |
| Copper | 0.5 mg ≦ Cu ≦ 0.67 mg | |
| Manganese | 0.67 mg ≦ Mn ≦ 1.67 mg | |
| Fluorine | 0.5 mg ≦ F ≦ 1.33 mg | |
| Cobalt | ≦0.05 | μg |
| Selenium | ≦23.33 | μg |

| | |
|---|---|
| Chromium | 16.67 μg ≦ Cr ≦ 66.67 μg |
| Molybdenum | 10 μg ≦ Mo ≦ 33.33 μg |

### Composition 3:

This composition, made for a person of more than 80 kg, exists as a powder to be diluted and as a liquid to be diluted in water up to 1.5 liters.

This composition is conditioned in a sachet and in a container for a liquid (flask) comprising:

Sachet

| | |
|---|---|
| NaCl | 2.19 g |
| Na₂SO₄ anh. | 8.52 g |
| KCl | 2 g ≦ KCl ≦ 4.5 g |
| NaHCO3 | 2.52 g |
| PEG 3350 | 88.5 g |

Flask

| Energy value | 1000 | kcal |
|---|---|---|
| Carbohydrates | 251 | g |
| Glucose | 8.1 | g |
| Maltose | 31.8 | g |
| Polysaccharides | 209.7 | g |
| Water | 250 | g |
| Electrolytes | | |
| Ca²⁺ | 0.06 g ≦ Ca²⁺ ≦ 0.16 g | |
| Mg²⁺ | 0.032 g ≦ Mg²⁺ ≦ 0.13 g | |
| PO₄³⁻ | 0.2 g ≦ PO₄³⁻ ≦ 0.4 g | |
| Food additive | | |
| Citric acid | 1.35 | g |
| Proteins | 20 | g |
| Trace elements | | |
| Iron | 3.33 mg ≦ Fe ≦ 5 mg | |
| Zinc | 3 mg ≦ Zn ≦ 4.67 mg | |
| Copper | 0.5 mg ≦ Cu ≦ 0.67 mg | |
| Manganese | 0.67 mg ≦ Mn ≦ 1.67 mg | |
| Fluorine | 0.5 mg ≦ F ≦ 1.33 mg | |
| Cobalt | ≦0.05 | μg |
| Selenium | ≦23.33 | μg |

| | |
|---|---|
| Chromium | 16.67 μg ≦ Cr ≦ 66.67 μg |
| Molybdenum | 10 μg ≦ Mo ≦ 33.33 μg |

### Composition 4:

This composition is a combination of electrolyte-PEG solution and Nutrical® as nutritive solution. The (electrolyte-)PEG solution can be any or a combination of the above described compositions 1, 2 and/or 3 made soluble in the above described clear liquid from the state of the art. Nutrical® (Nutricia Advanced Medical Nutrition, Belgium) is a carbohydrate beverage rich in energy with low water (moisture) and mineral content. The use of this carbohydrate beverage is recommended for people with insufficient energy intake or with an elevated need for energy. The carbohydrate beverage as described above is being sold in carton-based packages of 200 ml. Ingredients of this carbohydrate beverage are: demineralized water, glucose syrup, maltose, dextrin, acidity regulator (citric acid).

### The Carbohydrate Beverage as Described Above's Average Analysis Per 100 ml:

| | | |
|---|---|---|
| Energy value | 247 | kcal |
| Carbohydrates | 61.9 | g |
| Glucose | 2.2 | g |
| Maltose | 8.4 | g |
| Polysaccharides | 50.7 | g |
| Other | 0.5 | g |
| Water | 61 | g |

Electrolytes or minerals

| | | |
|---|---|---|
| Na | <5 | mg |
| K | <5 | mg |
| Cl | <5 | mg |
| Ca | <5 | mg |
| P | <5 | mg |
| Mg | <5 | mg |

The above described compositions 1, 2 & 3 can optionally comprise <10 g ascorbic acid (i.e. vitamin C).

### Example of Use

### Example 1

In order to prepare the digestive tract to an endoscopic capsule examination where the quality of the preparation has to be good to excellent (grade 1 or 2 according to Eliakim et al.), the patient has to follow the following administration:
In the evening two days before the examination, a sachet of composition 1 is made soluble in 1.5 liters of water and is administered to the patient.
The day before the examination, three sachets of composition 1 are made soluble in 3 times 1.5 liters of water and are administered to the patient during the whole day.
In the morning of the examination, a sachet of composition 1 is made soluble in 1.5 liters and is administered to the patient.

This allows the patient to feed exclusively on this composition starting from two days before the examination. He/she should therefore not take in additional liquid.

### Example 1 Bis

In order to prepare the digestive tract to an endoscopic capsule examination where the quality of the preparation has to be good to excellent (grade 1 or 2 according to Eliakim et al.), the patient has to follow the following administration:
In the morning and at noon two days before the examination, a contact laxative (e.g.: Senna Alexandrina syrup) is administered to the patient.
In the evening two days before the examination, a sachet of composition 1 is made soluble in 1.5 liters of water and is administered to the patient.
The day before the examination, three sachets of composition 1 are made soluble in 3 times 1.5 liters of water and are administered to the patient during the whole day.
In the morning of the examination, a sachet of composition 1 is made soluble in 1.5 liters and is administered to the patient.

This allows the patient to feed exclusively on this composition starting from two days before the examination. He/she should therefore not take in additional liquid.

### Example 2

In order to prepare the digestive tract for a radiological examination (ColoScan) where the quality of the preparation has to be excellent or good according to Vanner et al., the patient follows the following procedure:
One day before the examination, two sachets of the composition 1 are made soluble in two times 1.5 liters of water and are administered to the patient.
In the morning of the examination, a sachet of composition 1 is made soluble in 1.5 liters and is administered to the patient.

This allows the patient to feed exclusively on this composition starting from the day before the examination. He/she should therefore not take in additional liquid.

### Example 3

In order to prepare the digestive tract for diagnostic examination with a colonoscope where the quality of the preparation has to be satisfactory, good or excellent according to Vanner et al., the patient follows the following procedure:
One day before examination, two amounts of the composition 2 are made soluble in two times 1.5 liters of water and are administered to the patient.
In the morning of the examination, an amount of composition 2 is made soluble in 1.5 liters and is administered to the patient.

This allows the patient to feed exclusively on this composition starting from the day before the examination. He/she should therefore not take in additional liquid

### Example 4

In order to prepare the digestive tract for a therapeutic intervention where the quality of the preparation has to be satisfactory, good or excellent according to Vanner, the patient follows the following procedure:
The day before the examination, two amounts of composition 3 are made soluble in two times 1.5 liters of water and are administered to the patient.
In the morning of the examination, an amount of composition 3 is made soluble in 1.5 liters and is administered to the patient.

This allows the patient to feed exclusively on this composition starting from the day before the examination. He/she should therefore not take in additional liquid.

### Example 5

In order to prepare the digestive tract for PillCam™0 Colon 2 (Given Imaging Ltd., Israel) where the quality of the (oral bowel) preparation has to be acceptable, preferably good or more preferably excellent according to Vanner, the patient follows the following procedure:
Two days before the examination subject will have:
   base line serum electrolyte or ions and blood sugar or glucose level or glyc(a)emic index determined via collection of blood and
   one liter of PEG solution in the evening, water (or any other of the above described clear liquids from the state of the art) at will.
One day before the examination, composition 4 is administered to the patient according to the following schedule:
   One liter PEG and 200 ml of the carbohydrate beverage of composition 4 in the morning,
   400 ml of the carbohydrate beverage of composition 4 in the afternoon and
   one liter PEG and 200 ml of the carbohydrate beverage of composition 4 in the evening.
On this day there is a total intake of about 2000 kcal: 2(2×247 kcal)+(4×247 kcal)=1976 kcal. One meal equals about 700 kcal.
Nil by mouth (to fast a patient) except water (or any other of the above described clear liquids from the state of the art) at will.
Day of the examination subject will have:
   base line serum electrolyte or ions and blood sugar or glucose level or glyc(a)emic index determined via collection of blood and
   one liter of PEG solution after ingestion of the proprietary endoscopic capsule of the 1^{st} paragraph of example 5.

The above described (electrolyte-)PEG (solution) can be any or a combination of the above described compositions 1, 2 and/or 3 made soluble in the above described clear liquid from the state of the art.

Healthy volunteers are subjected to the procedure of example 5 after which physicians review the generated RAPID video. The technology behind the endoscopic capsule is that it contains an imaging device and light-source and it transmits images to the sensor array attached to the patient's abdomen and to the data recorder attached to a belt around the patient's waist. Images for review and diagnosis are downloaded by the physician to the Given workstation, on which the proprietary RAPID software is installed. Grading is done using a scale for each segmental section of the colon and overall for the entire video, an excellent grade is given when there is no more than small bits of adherent feces, a poor grade when there is a large amount of fecal residue. The objectives of this clinical trial or study are a.o.: test the hypothesis that carbohydrate oral rehydration in combination with PEG would increase the patient tolerance to the bowel preparation, evaluate efficiency in colon cleansing (or the colon cleansing level) of combination of electrolyte-PEG solution and the carbohydrate beverage of composition 4 for proprietary endoscopic capsule of the 1^{st} paragraph of example 5, to evaluate patient adherence to the combined regimen, to evaluate patient tolerance to the combined regimen, to evaluate the effect of the regimen on capsule transient time, . . .

1. An energetic or nutritive composition comprising:
at least one compound having laxative properties;
mineral salts (or electrolytes)preferably comprising sodium, potassium, chlorine, calcium, magnesium, phosphor; and/or
carbohydrates which may be assimilated by the human digestive system such as glucose, galactose, fructose, maltose, saccharose, polysaccharides.

2. The energetic or nutritive composition according to claim 1 having an energy value larger than or equal to 250 kcal.

3. The energetic or nutritive composition according to claim 1 having an energy value larger than or equal to 500 kcal.

4. The energetic or nutritive composition according to claim 1 having an energy value larger than or equal to 1000 kcal.

5. The energetic or nutritive composition according to claim 1 wherein the compound having laxative properties belongs to the class of osmotic laxatives.

6. The energetic or nutritive composition according to claim 5 wherein the compound having laxative properties is polyethylene glycol (PEG) the average molecular weight of which is larger than or equal to 3000 kDa.

7. The energetic or nutritive composition according to claim 6 wherein the compound having laxative properties is polyethylene glycol (PEG) for which the average molecular weight is comprised between 3000 kDa and 20000 kDa.

8. The energetic or nutritive composition according to claim 5 wherein the compound having laxative properties is polyethylene glycol (PEG) the average molecular weight of which is 3350 kDa.

9. The energetic or nutritive composition according to claim 1 which comprises trace elements.

10. The energetic or nutritive composition according to claim 1 which comprises proteins.

11. The energetic or nutritive composition according to claim 1 which comprises vitamins such as ascorbic acid.

12. A kit comprising:
either at least one sachet or flask containing the composition according to claim 1; or
at least one sachet or flask containing a laxative, at least one sachet or flask containing one or more mineral salts and at least one sachet or flask containing carbohydrates; forming a composition according to claim 1; or
at least one sachet or flask containing a laxative and at least one sachet or flask containing one or more mineral salts and carbohydrates; forming a composition according to claim 1.
